(19) United States
(12) Patent Application Publication
(10) Pub. No.: US 2005/0196761 A1
(43) Pub. Date: Sep. 8, 2005

(54) Array hybridization apparatus and method

(76) Inventor: Allen C. Thompson, San Jose, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/796,488

(22) Filed: Mar. 8, 2004

(51) Int. Cl. C12Q 1/68; C12M 1/34

(52) U.S. Cl. 435/6; 435/287.2

(57) ABSTRACT

An array hybridization apparatus and method. The apparatus employs a substrate backing, a slide, a spacer, and a gasket. In certain embodiments a lever and one or more tabs may be employed. The invention allows for more easily separating an array slide from a substrate backing. Methods of disassembly of the apparatus are also disclosed.

### FIELD OF THE INVENTION

The invention relates to the field of micro arrays, and more particularly to novel substrate backings for use with microarrays. In particular, the invention relates to an apparatus for helping to separate a microarray slide from a substrate backing.

### BACKGROUND OF THE INVENTION

Polynucleotide arrays (such as DNA or RNA arrays) are known and are used, for example, as diagnostic or screening tools. Such arrays include regions of usually different sequence polynucleotides arranged in a predetermined configuration on a substrate backing. These regions (sometimes referenced as “features”) are positioned at respective locations (“addresses”) on the substrate backing. In use, the arrays, when exposed to a sample, will exhibit an observed binding or hybridization pattern. This binding pattern can be detected upon interrogating the array. For example, all polynucleotide targets (for example, DNA) in the sample can be labeled with a suitable label (such as a fluorescent dye), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated by depositing previously obtained biopolymers (such as from synthesis or natural sources) onto a substrate backing, or by in situ synthesis methods. Methods of depositing obtained biopolymers include dispensing droplets to a substrate backing from dispensers such as pin or capillaries (such as described in U.S. Pat. No. 5,807,522) or such as pulse jets (such as a piezoelectric inkjet head, as described in PCT publications WO 95/25116 and WO 98/41531, and elsewhere). For in situ fabrication methods, multiple different reagent droplets are deposited from drop dispensers at a given target location in order to form the final feature (hence a probe of the feature is synthesized on the array stubstrate). The in situ fabrication methods include those described in U.S. Pat. No. 5,449,754 for synthesizing peptide arrays, and described in WO 98/41531 and the references cited therein for polynucleotides. The in situ method for fabricating a polynucleotide array typically follows, at each of the multiple different addresses at which features are to be formed, the same conventional iterative sequence used in forming polynucleotides from nucleoside reagents on a support by means of known chemistry. This iterative sequence is as follows: (a) coupling a selected nucleoside through a phosphite linkage to a functionalized support in the first iteration, or a nucleoside bound to the substrate backing (i.e. the nucleoside-modified substrate backing) in subsequent iterations; (b) optionally, but preferably, blocking unreacted hydroxyl groups on the substrate backing bound nucleoside; (c) oxidizing the phosphite linkage of step (a) to form a phosphate linkage; and (d) removing the protecting group (“deprotection”) from the now substrate backing bound nucleoside coupled in step (a), to generate a reactive site for the next cycle of these steps. The functionalized support (in the first cycle) or deprotected coupled nucleoside (in subsequent cycles) provides a substrate backing bound moiety with a linking group for forming the phosphite linkage with a next nucleoside to be coupled in step (a). Final deprotection of nucleoside bases can be accomplished using alkaline conditions such as ammonium hydroxide, in a known manner.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643. EP 0294196, and elsewhere.

Substrate backings used for microarrays are important because they enclose the polynucleotides used for the hybridizations. A variety of backings have been proposed for both deposition and in situ microarrays. A variety of materials have been used and proposed. For instance, the standard backing may comprise a glass substrate backing or similar type material. A typical gasket and/or spacer is then disposed onto the glass, adhered to the glass, or may be pre-cut and attached to the glass. The gaskets are designed to provide spacing so that the polynucleotides reside in a region defined as a hybridization chamber. However, a number of problems exist using glass backings for microarrays. One major problem regards the need for improved methods and devices to help in separating the microarray slide from the backing before or after readings have been taken. Often times the microarray slide will bond to the backing. Forces then applied to separate the backing from the microarray slide may cause the solution to be lost or the microarrays to be destroyed or damaged. Therefore, there is a substantial need to provide an improved apparatus and method for separation of microarray slides from backings.

It, therefore, would be desirable to provide an array hybridization apparatus that meets the above described needs and is easy to assemble and disassemble. It would also be desirable to provide an array hybridization apparatus in which the mode and parts for disassembling the array hybridization apparatus are self contained.

### SUMMARY OF THE INVENTION

The invention provides an array hybridization apparatus and method of making and disassembling the same. The array hybridization apparatus comprises a slide for holding an array, a substrate backing opposite the slide, a gasket interposed between the slide and the substrate backing, and a spacer. The spacer is interposed between the slide and the substrate backing adjacent to the gasket wherein when a force is applied to the substrate backing and the slide a portion of the slide separates from the substrate backing. The spacer may be positioned on a rotatably mounted lever or interposed between the slide and the substrate backing adjacent to the gasket to define a space between the slide, the substrate backing, the gasket and the spacer, when the slide and the substrate backing contact the gasket and the spacer. The spacer acts as a pivot point for separating the slide from the substrate backing.

The invention also provides a method for disassembling an array hybridization apparatus. The method comprises contacting a slide to a backing having a spacer wherein the spacer defines a space between the slide and the substrate backing; and applying a force to the slide to separate a portion of the slide from the substrate backing.

### BRIEF DESCRIPTION OF THE DRAWINGS

Embodiments of the invention will now be described with reference to the drawings, in which:

FIG. 1 illustrates a slide carrying an array, of the present invention;

FIG. 2 is an enlarged view of a portion of FIG. 1 showing ideal spots or features;

FIG. 3 is an enlarged illustration of a portion of the substrate backing in FIG. 2;

FIG. 4A is a perspective view of a first embodiment of the invention;

FIG. 4B is a plan view of a first embodiment of the present invention;

FIG. 4C is a cross section of a first embodiment of the present invention;

FIG. 4D is a cross section showing another embodiment of the present invention;

FIG. 5 is a perspective view of a second embodiment of the invention;

FIG. 6A is a perspective view of a third embodiment of the present invention;

FIG. 6B is a cross section view of the third embodiment of the present invention.

### DETAILED DESCRIPTION OF THE INVENTION

Before describing the invention in detail, it must be noted that, as used in this specification and the appended claims, the singular forms “a,” “an,” and “the” include plural referents unless the context clearly dictates otherwise. Thus, for example, reference to “a backing” includes more than one “backing”. Reference to a “spacer” or “substrate backing” includes more than one “spacer” or “substrate backing”. In describing and claiming the present invention, the following terminology will be used in accordance with the definitions set out below.

A “biopolymer” is a polymer of one or more types of repeating units. Biopolymers are typically found in biological systems (although they may be made synthetically) and particularly include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A “peptide” is used to refer to an amino acid multimer of any length (for example, more than 10, 10 to 100, or more amino acid units). A biomonomer fluid or biopolymer fluid reference a liquid containing either a biomonomer or biopolymer, respectively (typically in solution).

A “set” or “sub-set” of any item (for example, a set of features) may contain one or more than one of the item (for example, a set of clamp members may contain one or more such members). An “array”, unless a contrary intention appears, includes any one, two or three dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate backing at the various regions. However, either of the “target” or “target probes” may be the one that is to be evaluated by the other (thus, either one could be an unknown mixture of polynucleotides to be evaluated by binding with the other). An “array layout” refers collectively to one or more characteristics of the features, such as feature positioning, one or more feature dimensions, and some indication of a moiety at a given location. “Hybridizing” and “binding”, with respect to polynucleotides, are used interchangeably. When one item is indicated as being “remote” from another, this is referenced that the two items are at least in different buildings, and may be at least one mile, ten miles, or at least one hundred miles apart.

The term “adjacent” or “adjacent to” refers to a component or element that is near, next to or adjoining. For instance, a gasket may be adjacent to a spacer.

The term “substantially deformable”, “compressible” or “deformable” shall all have a similar meaning.

The term “slide” refers to any number of materials having at least one planar surface capable of contacting a gasket or spacer. The term shall be broad based to include substrate backings, polymeric materials, silica based materials, plastics etc. It's important that the “slide” maintain a certain amount of rigidity to compress or deform the gasket and contact the spacer. In certain instances a “slide” will be transparent to allow light to pass through its medium. However, this is not required. Also, the “slide” must be capable in certain instances to allow for the mounting or construction of an array on its surface. Although in certain cases this will not be required if the array is constructed on a separate surface.

The term “substrate backing” refers to any number of materials that maintain a rigid structure. For instance, materials may comprise plastic, metal, polypropylene, styrene, etc. A substrate backing may also comprise materials capable of being molded to a desired shape or design. For instance, thermoplastic materials may be employed. Other materials known in the art may also be employed.

It will also be appreciated that throughout the present application, that words such as “front”, “rear”, “back”, “leading”, “trailing”, “top”, “upper”, and “lower”, are all used in a relative sense only. “Fluid” is used herein to reference a liquid. Reference to a singular item, includes the possibility that there are plural of the same items present. Furthermore, when one thing is “slid” or “moved” or the like, with respect to another, this implies relative motion only such that either thing or both might actually be moved in relation to the other.

All patents and other cited references are incorporated into this application by reference.

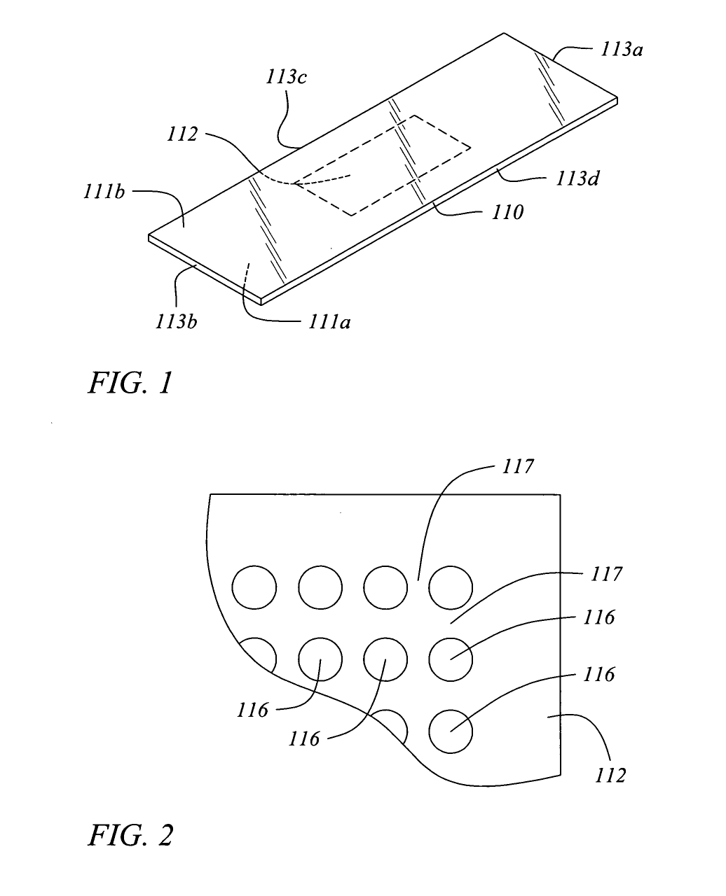

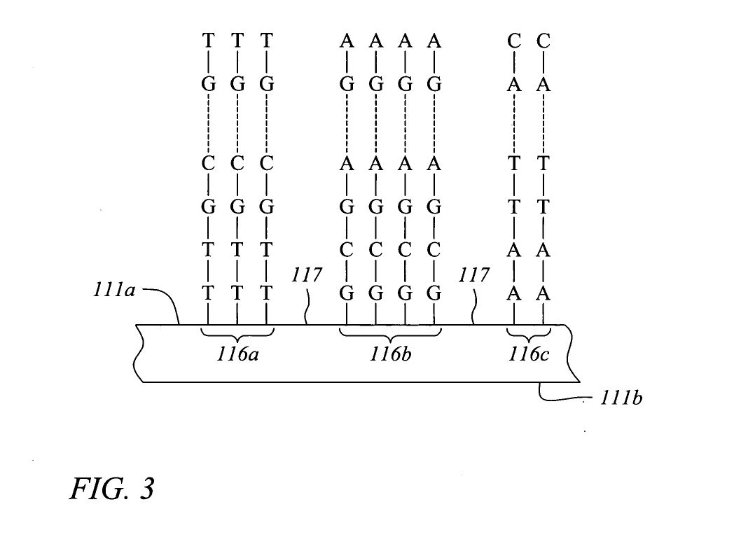

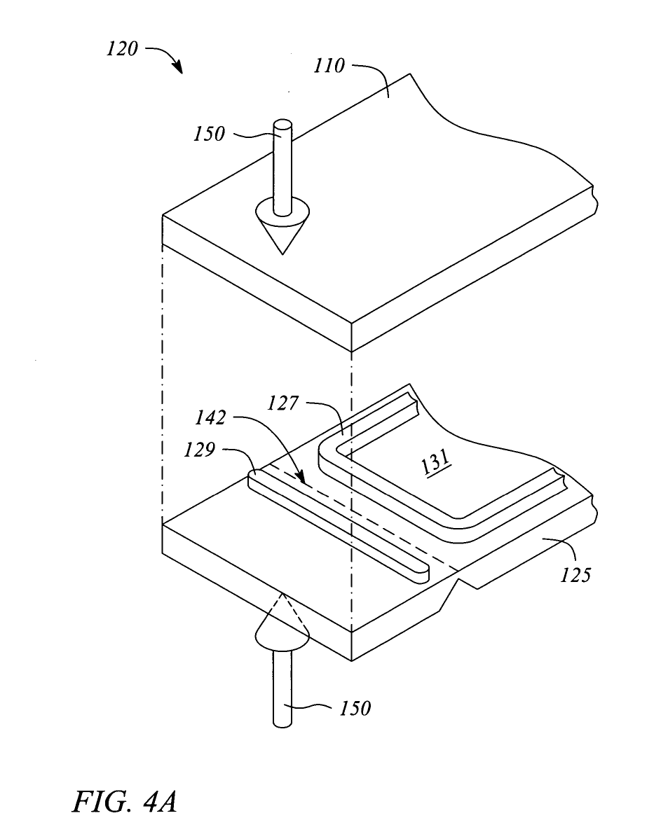

Referring first to FIGS. 1-3, typically the methods and apparatus of the present invention generate or use a contiguous planar transparent slide 110 carrying an array 112 disposed on a rear surface 111a of a substrate backing 110. It will be appreciated though, that more than one array (any of which are the same or different) may be present on the rear surface 111a, with or without spacing between such arrays. Note that one or more of the arrays 112 together will cover the entire region of the rear surface 111a, with regions of the rear surface 111a adjacent to the opposed sides 113c, 113d and the leading end 113a and the trailing end 113b of the slide 110. A front surface 111b of the slide 110 does not carry any of the arrays 112. Each of the arrays 112 can be designed for testing against any type of sample, whether a trial sample, reference sample, a combination of them, or a known mixture of polynucleotides (in which latter case the arrays may be composed of features carrying unknown sequences to be evaluated). The slide 110 may be of any shape, and any holder used with it adapted accordingly, although the slide 110 will typically be rectangular in practice. The array 112 contains multiple spots or features 116 of biopolymers in the form of polynucleotides. A typical array may contain from more than ten, more than one hundred, more than one thousand or ten thousand features, or even more than from one hundred thousand features. All of the features 116 may be different, or some or all could be the same. In the case where the array 112 is formed by the conventional in situ or deposition of previously obtained moieties, as described above, by depositing for each feature at least one droplet of reagent such as by using a pulse jet such as an inkjet type head, interfeature areas 117 will typically be present which do not carry any polynucleotide. It will be appreciated though, that the interfeature areas 117 could be of various sizes and configurations. Each feature carries a predetermined polynucleotide (which includes the possibility of mixtures of polynucleotides). As per usual, A, C, G, T represent the usual nucleotides. It will be understood that there may be a linker molecule (not shown) of any known types between the rear surface 111a and the first nucleotide.

The slide 110 may also carry on the front surface 111b, an identification code in the form of a bar code 115 printed on an opaque substrate backing in the form of a paper label attached by adhesive to the front side 111a (not shown in FIGS.). By “opaque” in this context is referenced that the means used to read the bar code 115 (typically a laser beam) can not read the bar code 115 through the label without reading errors. Typically this means that less than 60% or even less than 50%, 30%, 20% or 10% of the signal from the code passes through the substrate backing. The bar code 115 contains an identification of the array 112 and either contains or is associated with, array layout or layout error information in a manner such as described in U.S. patent applications.

For the purpose of the discussions below, it will be assumed (unless the contrary is indicated) that the array 112 is a polynucleotide array formed by the deposition of previously obtained polynucleotides using pulse jet deposition units. However, it will be appreciated that an array of other polymers or chemical moieties generally, whether formed by multiple cycles in situ methods adding one or more monomers per cycle, or deposition of previously obtained moieties, or by other methods, may be present instead.

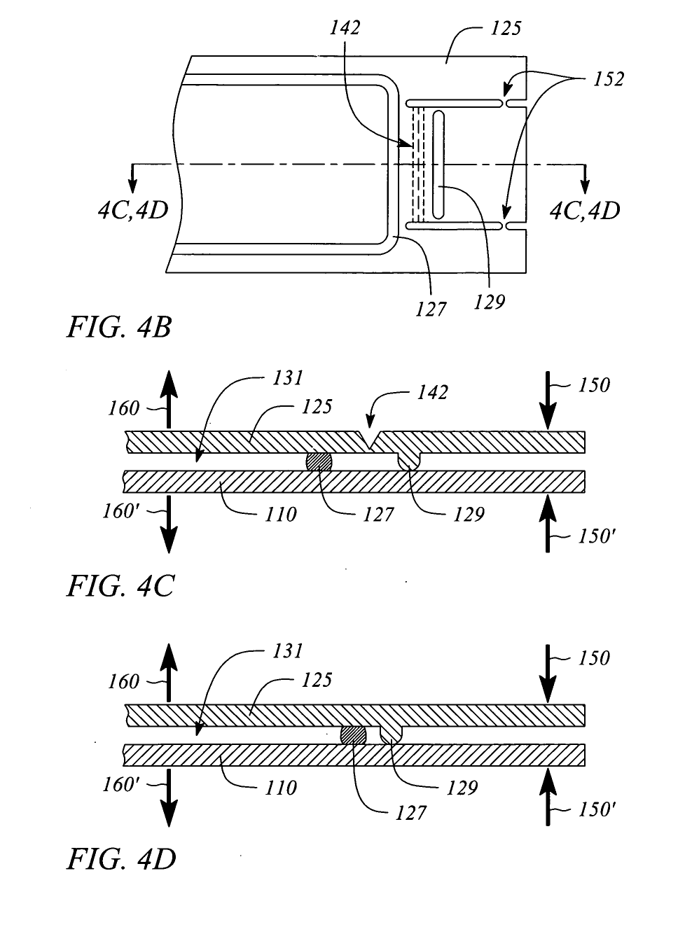

Referring now to FIGS. 4A-4D, the first embodiment of the invention comprises a slide 110, a gasket 127, a spacer 129, and a substrate backing 125. An optional living hinge 142 may be employed on the substrate backing 125 or the slide 110 (See FIG. 4C). FIG. 4A shows the living hinge 142 positioned or designed in the substrate backing 125. The living hinge 142 aids in the separation of the backing 125 from the slide 110 when a force 150 or 150′ is applied to the substrate backing 125 and the slide 110. In most cases, the spacer 129 is positioned at least 1-5 centimeters from the edge of the substrate backing 125 or slide 110 adjacent to the gasket 127 and the living hinge 142. If the spacer is too close to the end of the slide 110 or substrate backing 125 it will not be effective in acting as a pivot point to separate the slide 110 from the substrate backing 125. In addition if it is positioned too far away from the edge of the slide 110 or substrate backing 125, the slide 110 will not be appropriately balanced over the substrate backing 125. When a force 150 and/or 150′ is/are applied to the slide 110 and the substrate backing 125 the spacer 129 acts as a pivot point for the slide 110 that causes the second edge of the slide 110 to separate from the substrate backing 125 (See FIG. 4C and 4D. Separation not shown in the drawing). This then allows for ease of removal of the substrate backing 125 from the slide 110. FIG. 4B shows a plan view of the same embodiment of the invention. The figure more clearly shows how the spacer 129 is positioned on the substrate backing 125.

The slide 110 may typically contain or be attached to the array 112 and may comprise any number of transparent materials such as glass, plastic, silicon or other materials known in the art to contain or be capable of containing arrays. Slide 110 can be thought of as the array substrate backing, but need not contain the array 112. The array 112 could also be attached or part of the substrate backing 125. The slide 110 may be designed in a variety of shapes, sizes and widths.

The substrate backing 125 may be thought of as being the backing for the hybridization apparatus 120. However, in certain embodiments the substrate backing 125 may actually contain or comprise the array 112. The substrate backing 125 may be designed in a variety of shapes, sizes and widths. The material may allow for molding the material to a variety of shapes and designs. In addition, the gasket 127 can be molded in place or may comprise a portion of the substrate backing 125. The material may allow for a more efficient design of the gaskets 127 as well as a more efficient construction process for the array hybridization apparatus 120. For instance, the gasket 127 may comprise a portion of the substrate backing 125 and may be constructed using injection molding at the time of construction of the substrate backing 125. An injection molded substrate backing 125 and gasket 127 may provide for more efficient use of the space across the substrate backing 125 to allow more features per unit area on the substrate backing 125. In addition, the injection molding allows for more accurate construction as well as less steps in the construction of the array hybridization apparatus 120.

The gasket 127 may be attached to the slide 110, the substrate backing 125 or both and is designed for holding or retaining the hybridization solutions for the array 112. Typically, the gasket 127 will be rectangular in shape and will be attached to the substrate backing 125. The shape and design of the gasket 127 is not important to the invention. However, it is important to the invention that the gasket 127 maintains a sufficient compressibility so as to form a seal between the slide 110, the gasket 127 and the substrate backing 125 when they contact each other. The gasket 127 must also retain the hybridization solution when the slide 110, substrate backing 125, the gasket 127 and the spacer 129 are all contacted. The gasket 127 may comprise any number of materials that are substantially deformable. For instance, the gasket 125 may comprise materials such as rubber, silicon, silicone, acrylamides, polyacrylamides, non-synthetic polymers and synthetic polymers etc.

The spacer 129 may be attached to the slide 110, the substrate backing 125 or both. Typically, the spacer 129 will be attached to the slide 110 when the gasket 127 is attached to the substrate backing 125. The spacer 129 may comprise any number of shapes and sizes. It may also be positioned in any number of positions on the substrate backing 125 or slide 110 and may comprise substantially non deformable or non-compressable materials such as metal, wood, plastic etc. For instance, the spacer 129 needs to be less deformable or compressible relative to the gasket 127. This allows the gasket 127 to act as a seal but deform only to the extent of the height of the spacer 129. Since the spacer 129 does not further collapse or compress the height or volume of the array hybridization chamber 131 can be gauged. The spacer 129 can range in height of from 25 to 500 microns. This forms the hybridization chamber 131 having a fixed volume based on the height of the spacer 129. The spacer 129 will similarly retain a height in the range of from 25 to 500 microns. In certain embodiments, the spacer 129 needs to be closely spaced to the gasket 127 and optional living hinge 142. For instance, if the spacer 129 is spaced too far away from the gasket 127 the living hinge 142 will not operated correctly. The spacer 129 will not act effectively as a pivot point. In addition, if the gasket 127 if spaced too close to the spacer 129 then the slide 110 will not be maintained above the substrate backing 125. This generally will be based on trial an error. However, generally, the spacer 129 should be spaced from the gasket 127 from about 1 to 5 centimeters.

FIG. 4B shows a plan view of an embodiment of the invention. The figure shows the relative positioning of the spacer 129 and the living hinge 142. Although the living hinge 142 is shown on the opposing surface of the substrate backing 125, it may be effectively employed on both surfaces (living hinge not shown on top surface in diagrams).

FIG. 4C shows a cross sectional view of an embodiment of the present invention. The figure more clearly shows the shape, design, and positioning of the living hinge 142 and how it is employed with the present invention. FIG. 4C is similar to FIG. 4B, but flipped up side down to shown how the forces 150 and 150′ create the forces 160 and 160′ to separate the slide 110 from the substrate backing 125.

FIG. 4D shows a similar embodiment to 4B and 4C, but without the use of the living hinge 142.

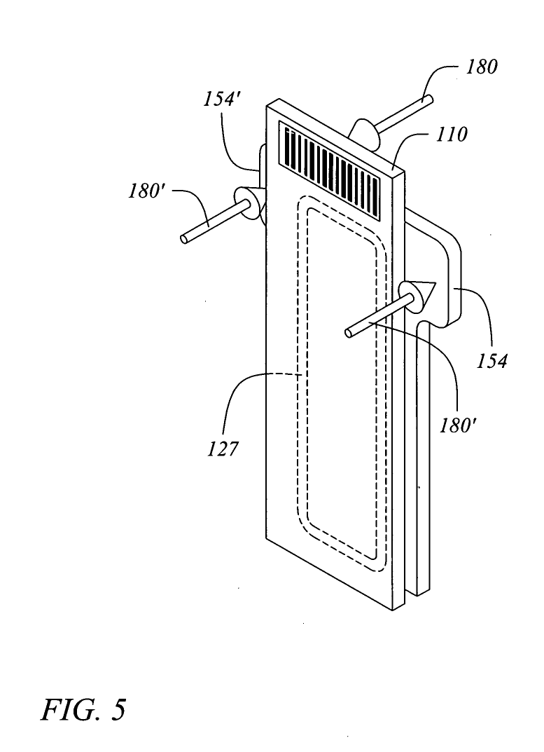

Referring now to FIG. 5, a second embodiment of the present invention is shown. In this embodiment of the invention one or more tabs 154 and 154′ may be employed on the substrate backing 125 and/or the slide 110. The tabs 154 and 154′ are employed to help in separating the substrate backing 125 from the slide 110. The tabs 154 and 154′ may comprise a variety of shapes, sizes and materials. As shown in the diagram the tabs 154 and 154′ extend away from either or both the substrate backing 125 and/or slide 110. The tabs 154 and 154′ may be shaped and designed in a variety of formats. The tabs 154 and 154′, however, must be large enough for a user to exert an opposing force to separate the substrate backing 125 from the slide 110. This force may be applied by the users fingers or any other methods that may be known or used in the art.

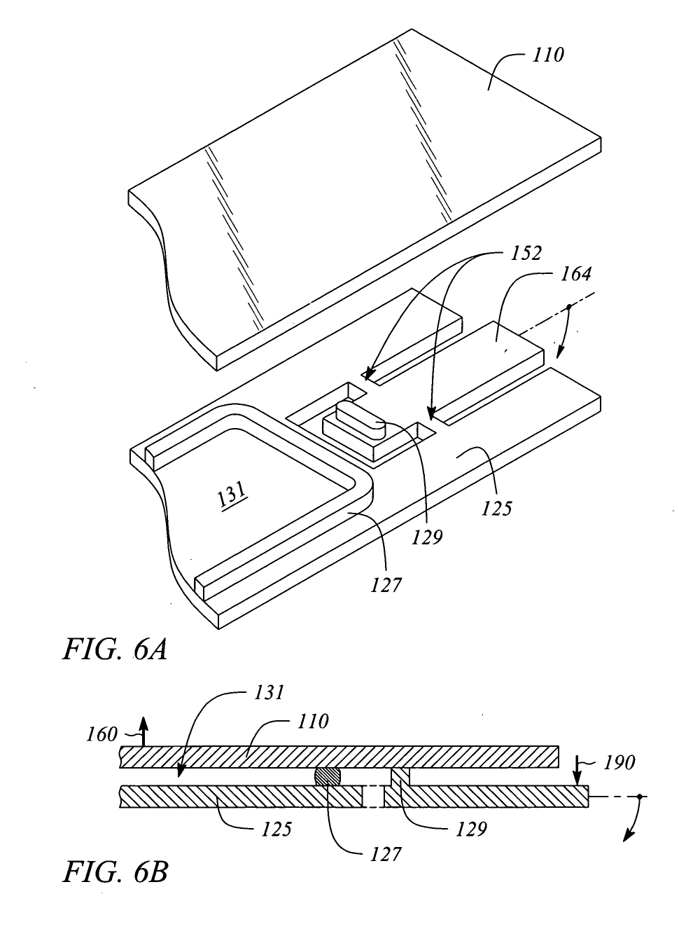

FIGS. 6A and 6B show a third embodiment of the present invention. In this embodiment of the invention, the spacer 129 is positioned on a rotatable lever 164. The rotatable lever 164 aids in the separation of the slide 110 from the substrate backing 125. In order for the slide 110 to be separated from the substrate backing 125, a force is applied to the rotatable lever 164. This causes the rotatable lever 164 to move and pivot about the hinge 152.

Having described the apparatus of the present invention, a description of the method of disassembly is now in order. Referring now to FIGS. 4A-4D, the slide 110 contacts the substrate backing 125 and the spacer 129. The slide 110 encloses the hybridization chamber 131 in the gasket 127 to create a seal. Readings are then taken by running the assembled apparatus through a micro array reader (not shown). In order to separate the slide 110 from the substrate backing 125, a force 150 or 150′ (See FIG. 4A) is applied to the edge of the slide 110 or the substrate backing 125. The substrate backing 125 separates from the slide 110 by the forces 150 and 150′ that are directed toward each other. These forces cause the slide 110 to separate from the substrate backing 125 because these inward forces 150 and 150′ are greater than the seal or bond between the slide 110 and the gasket 127 located on the substrate backing 125.

Referring now to FIGS. 4A-4D, a living hinge 142 may be employed to increase the amount and location of forces needed to separate the slide 110 from the substrate backing 125. In this embodiment of the invention a portion of substrate backing 125 acts as a lever, while the base of the living hinge 142 acts as a fulcrum. The fulcrum and living hinge 142 act to increase the overall forces to separate the substrate backing 125 from the slide 110. For instance, forces 150 and 150′ are applied towards each other on one end of the slide 110 and the substrate backing 125. The forces 150 and 150′ are applied in an inward direction and cause opposite forces 160 and 160′ at the opposing end of the slide 110 and the substrate backing 125 (See FIG. 4C-4D). This causes the slide 110 to separate from the substrate backing 125.

Referring now to FIG. 5, tabs 154 and 154′ are employed for separating the slide 110 from the substrate backing 125. The tabs 154 and 154′ may be on either or both of the slide 110 and the substrate backing 125. Opposite forces 180 and 180′ may be applied to the tabs to increase the space between them. The opposing forces cause the slide 110 to separate from the substrate backing 125.

Referring now to FIGS. 6A and 6B, the lever 164 is mounted for rotatable movement about the hinge 152. The spacer 129 is positioned on the lever 164. The slide 110 contacts the spacer 129 and the gasket 12. When a downward force 180 is applied to the lever 164, the spacer 129 acts as a pivot point and the slide 110 is separated from the gasket 127. This is accomplished by the opposing forces 160 and 160′ created by translation of the downward force 180 about the rotatably mounted hinge 152.

Clearly, minor changes may be made in the form and construction of the invention without departing from the scope of the invention defined by the appended claims. It is not, however, desired to confine the invention to the exact form herein shown and described, but it is desired to include all such as properly come within the scope claimed.

1. An array hybridization apparatus for separating a slide from a substrate backing, comprising:
(a) A slide for holding an array;
(b) a substrate backing being positioned opposite the slide;
(c) a gasket interposed between the slide and the substrate backing; and
(d) a spacer interposed between the slide and the substrate backing adjacent to the gasket wherein when a force is applied to the substrate backing and the slide a portion of the slide separates from the substrate backing.

2. An array hybridization apparatus as recited in claim 1, wherein the gasket comprises a deformable material.

3. An array hybridization apparatus as recited in claim 1, wherein the spacer comprises a substantially non-deformable material.

4. An array hybridization apparatus as recited in claim 1, wherein the gasket is attached to the slide.

5. An array hybridization apparatus as recited in claim 1, wherein the gasket is attached to the substrate backing.

6. An array hybridization apparatus as recited in claim 1, wherein the gasket comprises a portion of the substrate backing.

7. An array hybridization apparatus as recited in claim 1, wherein the gasket is attached to both the slide and the substrate backing.

8. An array hybridization apparatus as recited in claim 1, wherein the spacer is attached to the slide.

9. An array hybridization apparatus as recited in claim 1, wherein the spacer is attached to the substrate backing.

10. An array hybridization apparatus as recited in claim 1, wherein the spacer is attached to both the slide and the substrate backing.

11. An array hybridization apparatus as recited in claim 1, wherein the spacer comprises a material selected from the group consisting of polyurethanes, plastics, acrylics, metals and non-deformable or less deformable polymers.

12. An array hybridization apparatus as recited in claim 1, wherein the spacer is between 25 to 500 microns in height.

13. An array hybridization apparatus as recited in claim 11, wherein the array hybridization chamber is between 25 to 1000 microns in height.

14. A method of disassembling an array hybridization apparatus having a slide contacting a gasket and spacer, comprising:
a. applying a force to the edge of the slide to separate a portion of the slide from the substrate backing.

15. An array hybridization apparatus as recited in claim 1, further comprising a living hinge.

16. An array hybridization apparatus as recited in claim 1, further comprising a lever rotatably mounted on a hinge.
